(19) United States
(12) Patent Application Publication
(10) Pub. No.: US 2007/0276383 A1
(43) Pub. Date: Nov. 29, 2007

(54) Osteotomy system

(75) Inventor: John M. Rayhack, Tampa, FL (US)

Correspondence Address: HOLLAND & KNIGHT LLP;ATTN: STEFAN V. STEIN/ IP DEPT., POST OFFICE BOX 1288, TAMPA, FL, 33601-1288, US

(73) Assignee: Rayhack L.L.C.

(21) Appl. No.: 11/432,451

(22) Filed: May 11, 2006

(51) Int. Cl. A61F 2/30

(52) U.S. Cl. 606/69

(57) ABSTRACT

Improved bone plate having locking screw holes and non-locking cortical screw holes and a slot. Bone plate includes minimized thickness, width, and length while providing appropriate rigidity and strength necessary for treatment in ulnar shortening or other long bone shortening procedures.

### BACKGROUND

1. Technical Field

The present invention relates to medical devices useful in performing an Osteotomy or precision bone cutting procedure.

2. Background Information

An osteotomy of the ulna or other long bone is commonly used to treat numerous skeletal maladies. Ulnar shortening osteotomy procedures have been associated with complications such as malrotation, angulation, malunion, delayed union, and non-union, which typically result from inaccurate or non-parallel bone cuts, improper closure of the osteotomy gap, or inadequate bone fixation. Several procedures and systems have been developed to greatly reduce or entirely eliminate these complications. For example, U.S. Pat. No. 5,042,983 (“the '983 patent”), which is incorporated herein in its entirety by reference, discloses a precision bone cutting guide for precise perpendicular or angular cutting of a bone with a conventional bone saw. Once the bone is cut, as detailed in the '983 patent, a slotted plate is used to accurately and precisely secure the two bone portions.

Conventional slotted plates, however, have several limitations. In particular, to properly and securely join the two bone ends, the slotted plate must be able to withstand the forces that the ulna is typically subjected to during daily life activities. That is, the slotted plate must resist flexion, extension, and axial rotation stresses, which could lead to non-union of the bone and ultimate plate failure due to cycling. Moreover, conventional slotted plates must resist such stresses despite having a plurality of holes and a slotted portion, which all act to weaken the bone plate. Because of these requirements, many conventional slotted plates are made of stainless steel or titanium, and have a relatively thick, squared-off cross-section and a significant longitudinal dimension. Unfortunately, the thick, squared-off cross-section of the slotted plate often results in the plate being visible or palpable along the ulnar subcutaneous border. Among other things, this can be bothersome, painful, and/or unsightly to the patient. This often results in the need for a second operative procedure to remove the plate and screws. Additionally, the thickness of conventional bone plates often limits placement of the bone plate in difficult-to-access anatomical locations. As a result, the anatomical location of the procedures that can be performed is limited by many conventional bone plates.

### BRIEF SUMMARY

Accordingly, it is an object of the present invention to provide a medical device having features that resolve or improve upon one or more of the above-described drawbacks and limitations.

According to one aspect of the present invention, the foregoing object is obtained by providing an improved bone plate having a tapered proximal end, a tapered distal end, and a gently curved longitudinal portion extending between both ends. The improved bone plate also has a convex upper surface and a concave lower surface.

According to another aspect of the present invention, an improved plate is provided in which the gently curved longitudinal portion and two tapered ends have a top surface and a bottom surface. The bottom surface is shaped so as to mate with the contours of a bone surface, such as the ulnar bone surface. Additionally, the top and bottom surfaces are spaced apart a maximum distance between 0.119 in. and 0.113 in. in the middle of the gently curved longitudinal portion of the plate. A slot is also provided. The slot extends from the top surface to the bottom surface of the improved plate. Locking, machine-threaded screws are used to secure at least the proximal and distal tapered end portions of the improved plate. By using locking screws in conjunction with threaded screw holes in the locking plate, the overall height, length, and width dimensions of the bone plate can be substantially reduced.

### BRIEF DESCRIPTION OF THE DRAWINGS

FIG. 1 is a cross-sectional side view of one embodiment of an improved bone plate.

FIG. 2 is an end view of one embodiment of an improved bone plate as secured to an ulnar bone by bone screws.

FIG. 3 is a cross-sectional side view of one embodiment of an improved bone plate as secured to an ulnar bone by bone screws.

FIG. 4 is a top plan view of one embodiment of an improved bone plate.

FIG. 5 is a perspective side view of a low profile saw guide configured to be used with the improved bone plate of one embodiment of the present invention.

FIG. 6 is a perspective side view of a low profile saw guide and a straight drill guide configured to be used with the improved bone plate of one embodiment of the present invention.

FIG. 7 is a perspective side view of a saw blade to be used with a saw guide.

FIG. 8 is a perspective side view of a pair of plate benders configured to be used with the improved bone plate of one embodiment of the present invention.

FIG. 9 is a low profile compression device to be used with the improved bone plate of one embodiment of the present invention.

FIG. 10 is a perspective side view of a low profile angled drill guide configured to be used with the low profile compression device and the improved plate of one embodiment of the present invention.

FIG. 11 is a perspective side view of a combination drill bushing configured to be used with the angled drill guide and the improved plate of one embodiment of the present invention.

FIG. 12 is a perspective side view of a hand held drill guide configured to be used with the improved plate of one embodiment of the present invention.

FIG. 13 is a cross-sectional side view of a combination drill bushing configured to be used with the improved plate of one embodiment of the present invention.

FIG. 14 is a cross-sectional end view of a threaded locking screw configured to be used with the improved plate of one embodiment of the present invention.

### DETAILED DESCRIPTION OF THE DRAWINGS AND THE PRESENTLY PREFERRED EMBODIMENTS

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances details have been omitted, which are not necessary for an understanding of the present invention, such as conventional details of fabrication and assembly.

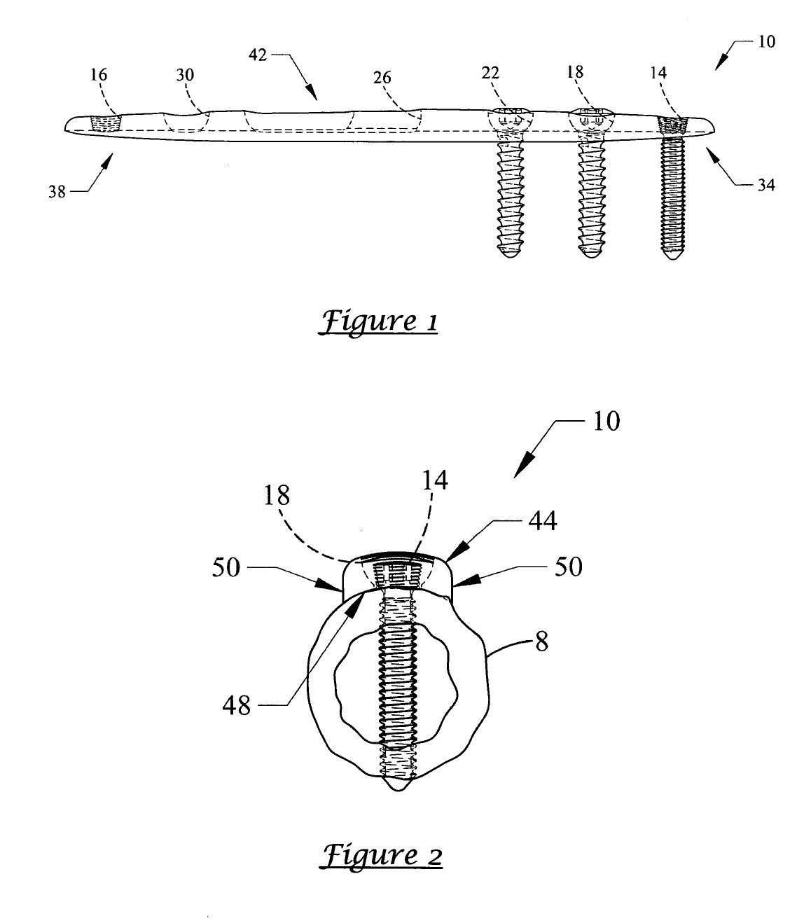

FIGS. 1-4 illustrate an embodiment of an improved bone plate used to join two ulnar bone segments cut or separated during an osteotomy procedure. In general, the illustrated embodiment includes a bone plate 10 having a proximal end portion 34, a distal end portion 38, and a gently curved longitudinal portion 42. Bone plate 10 further includes a number of openings or holes for receiving attachment screws. More specifically, bone plate 10 includes locking screw holes 14 and 16, as well as cortical screw holes 18, 22, 26, and 30.

As best illustrated in FIG. 1, bone plate 10 has an elongated configuration with tapered ends. The ends are tapered both as to top-to-bottom thickness and side-to-side width. Bone plate 10 can also be provided with a ramped portion disposed at one or both of the proximal or distal portions of the bone plate. As illustrated in FIG. 2, bone plate 10 includes a top surface 44 and a bottom surface 48. The top surface is generally convex to further reduce soft tissue trauma and palpability by the patient. The bottom surface 48 is generally shaped to mate with the corresponding surface of ulna bone 8 and can be placed on either the volar surface or subcutaneous surface of the ulna. Preferably, bone plate 10 is generally about 3.375 inches in length and 0.358 inches in maximum width, except that the width narrows at the distal and proximal end portions, as shown in FIG. 4. Bone plate 10 is generally about 0.119 inches in height.

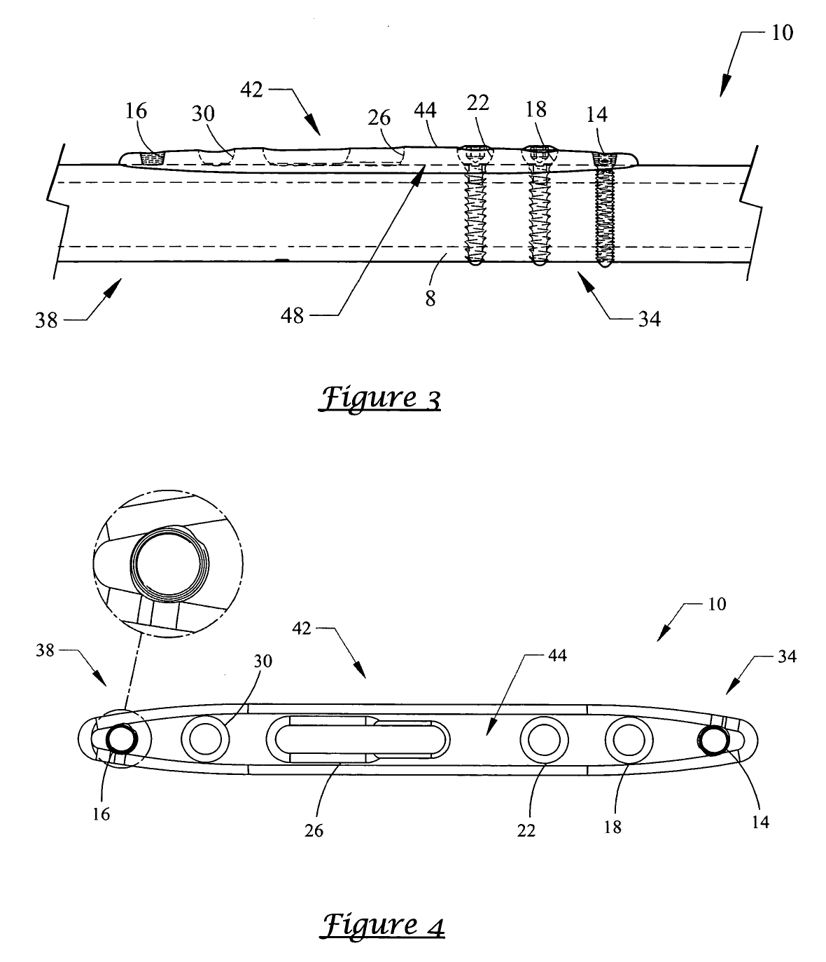

As illustrated in FIGS. 1,3 and 4, bone plate 10 includes a plurality of screw holes adapted to receive cortical screws. More specifically, the proximal end portion 34 includes a locking screw hole 14 that provides an internal female thread. The distal end portion 38 similarly includes a locking screw hole 16 having an internal female thread. Locking screw holes 14 and 16 are adapted to receive a machine-threaded locking screw between 2.7 mm and 3.0 mm in diameter and 12-20 mm in length. This screw length allows both cortices of the bone to be fully penetrated, as best illustrated in FIGS. 2 and 3. It should be noted that the pitch and diameter of locking screw holes 14 and 16 must typically correspond with the pitch and diameter of the threaded portion of the locking screws to be used with the bone plate.

It should further be noted that, in use, the locking screws are affixed to the bone plate so that the top surface of the locking screw is flush (or below) the top surface 44 of the locking bone plate. By using a locking screw, the screw head diameter can be reduced and thus the plate dimension can be correspondingly reduced. This also reduces potential soft-tissue trauma from protruding screw heads. It should be noted that, although only two locking screws are shown in the illustrated embodiment, additional locking screws can be used in place of the non-locking screws described below. In this case, the plate would need to be provided with corresponding threaded screw holes.

The use of locking screws provides greatly improved fixation between the bone plate and the bone. As a result of the improved fixation provided by the locking screws, the size of the bone plate can be greatly reduced while maintaining the necessary bone plate strength and rigidity. Moreover, the reduced-size bone plate decreases the potential for damage to the soft tissues surrounding the ulna. Examples of appropriate locking screws include screws with a variable thread pitch, and fully threaded design.

As best illustrated in FIGS. 1 and 3, the portion of bone plate 10 that is proximal to slot 26 further includes screw holes 18 and 22. Screw holes 18 and 22 are dimensioned to receive self-tapping cortical screws. Alternatively, non-self tapping screws can be used if the bone hole is first tapped by the surgeon. Screw holes 18 and 22 have a concave or chamfered configuration so that the cortical screws are as nearly flush with top surface 44 (FIG. 2) as possible. This chamfered configuration further reduces soft tissue trauma and palpability of the bone plate 10 and the screws used therewith. An additional screw hole 30 similar to screw holes 18 and 22 is provided distal to slot 26. Similarly, a cortical screw is secured through screw hole 30 to the ulna.

As best illustrated in FIG. 3, a fortified slot 26 is provided along the gently curved longitudinal portion 42. In use, slot 26 is aligned directly over the bone cut. More specifically, slot 26 is configured to receive a smaller diameter 2.7 mm cortical screw closest to the center of the plate and a 3.5 mm cortical screw (similar to screws 18 and 22) in the distal part of the slot. This arrangement further secures together the proximal and distal bone fragments. The cortical screws can be a self-tapping, or non-self tapping cortical screw as discussed above in relation to screw holes 18, 22, and 30.

The bone plate of the present invention can be formed or machined from a number of materials. The bone plate can be machined from surgical-quality alloys, including stainless steel. The bone plate can alternatively be formed from titanium.

Alternatively, the bone plate can be formed from an implantable grade polymer, biomaterial, or reabsorbable material. One exemplary polymer material is PEEK OPTIMA®, which is available from Invibio®. To form the bone plate from a material such as PEEK OPTIMA®, the polymer can be injection molded, compression molded, or extruded into the requisite bone plate shape. One exemplary reabsorbable material is a copolymer, such as the L-lactic acid and glycolic acid copolymer ReUnite®, which is available from Arthrotech, a Biomet Company. The bone plate can also be formed from high density plastics.

It should be noted that the screws used with the bone plate should be formed from the same material as the bone plate. That is, titanium screws should be used with a titanium bone plate, and stainless steel screws should be used with a stainless steel plate screws. Likewise, bioabsorbable screws should be used with a bioabsorbable plate.

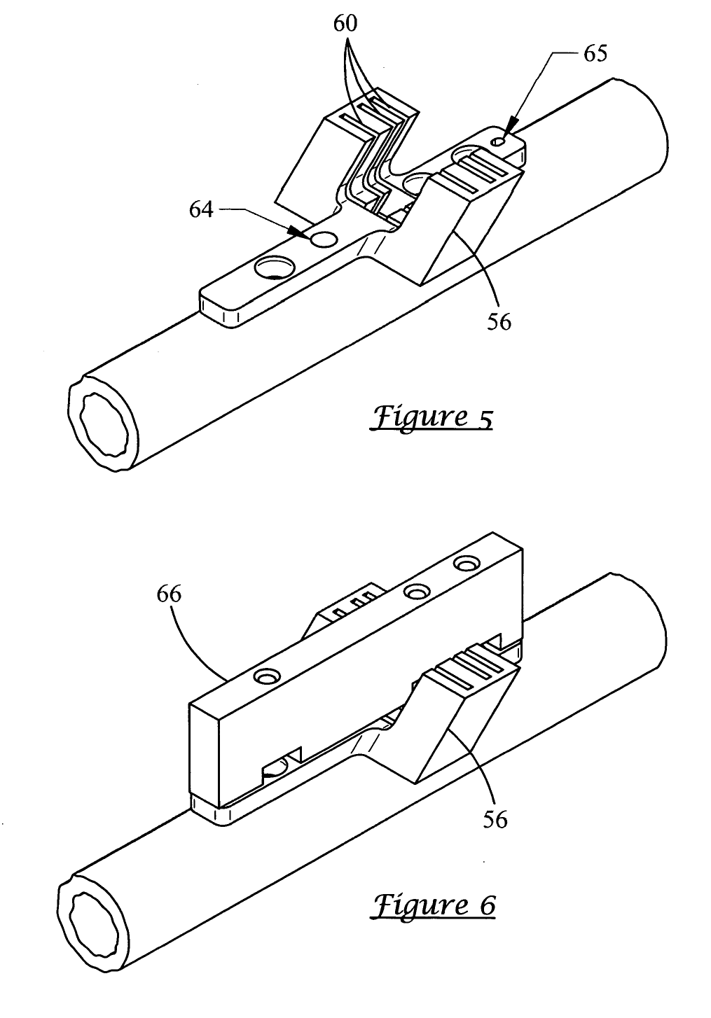

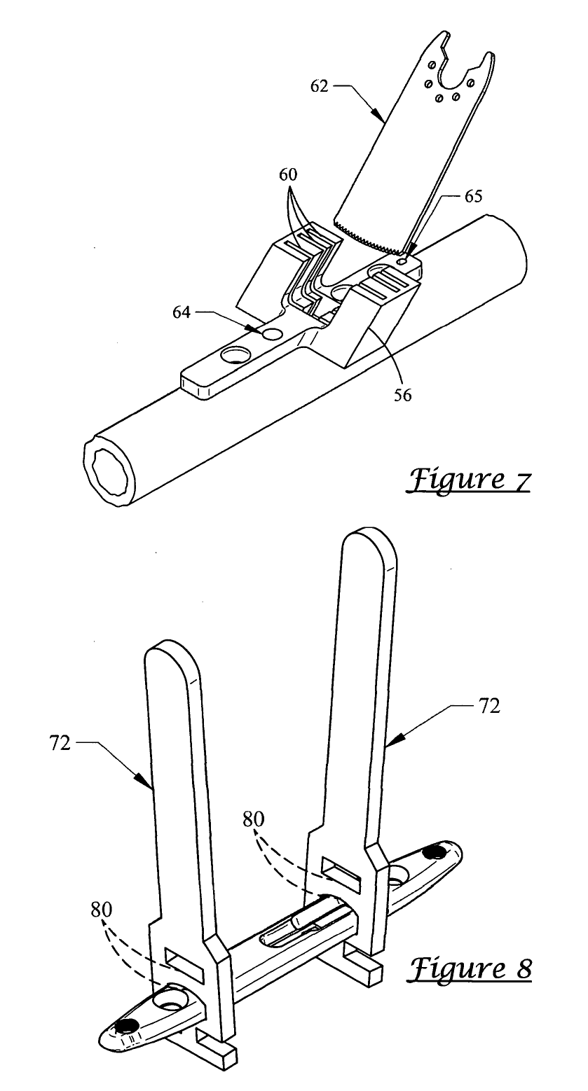

As shown in FIG. 5, a saw guide 56 is provided to complete a precision bone cut of the ulna in preparation for attachment of a bone plate. The saw guide 56 is provided with a plurality of saw guide parallel slots 60 and a drill guide channel 65 and a drill guide hole 64 for securing the straight drill guide to the saw guide. The saw guide 56 can be temporarily secured to the ulna with three 3.5 mm cortical screws by using a straight drill guide 66 (FIG. 6) or a standard hand held drill guide, as shown in FIG. 12, to pre-drill screw holes and then securing the saw guide 56 to the ulna with the screws. Once the saw guide 56 is secured, a saw and saw blade 62 can be used to cut through the ulna, as illustrated in FIG. 7.

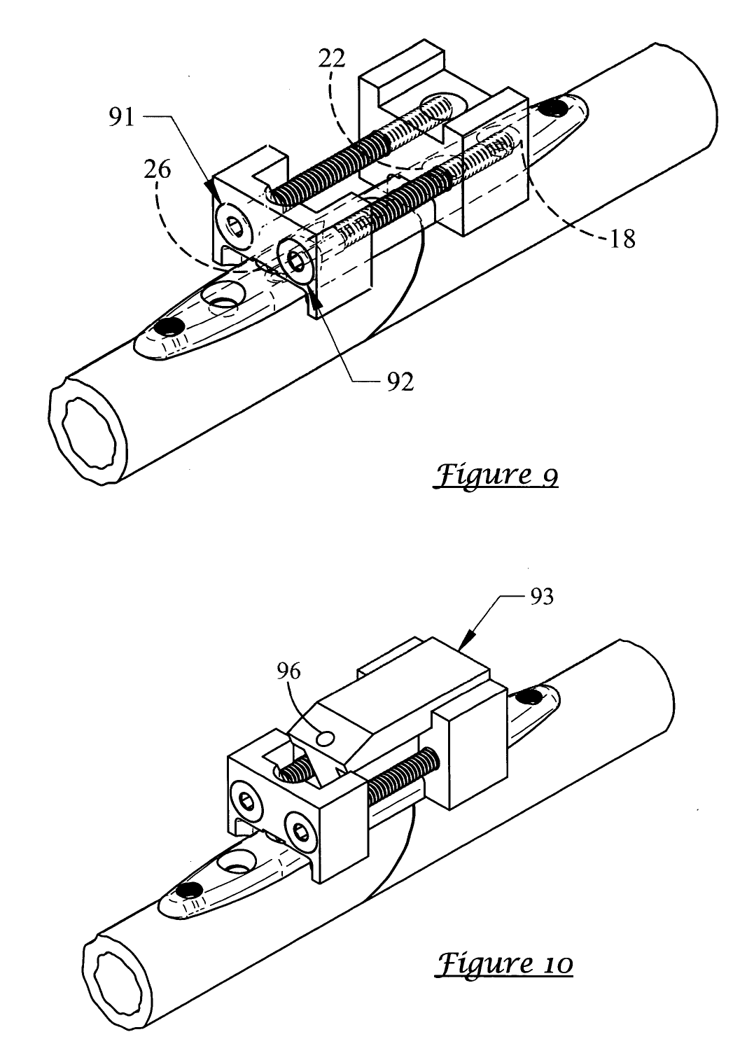

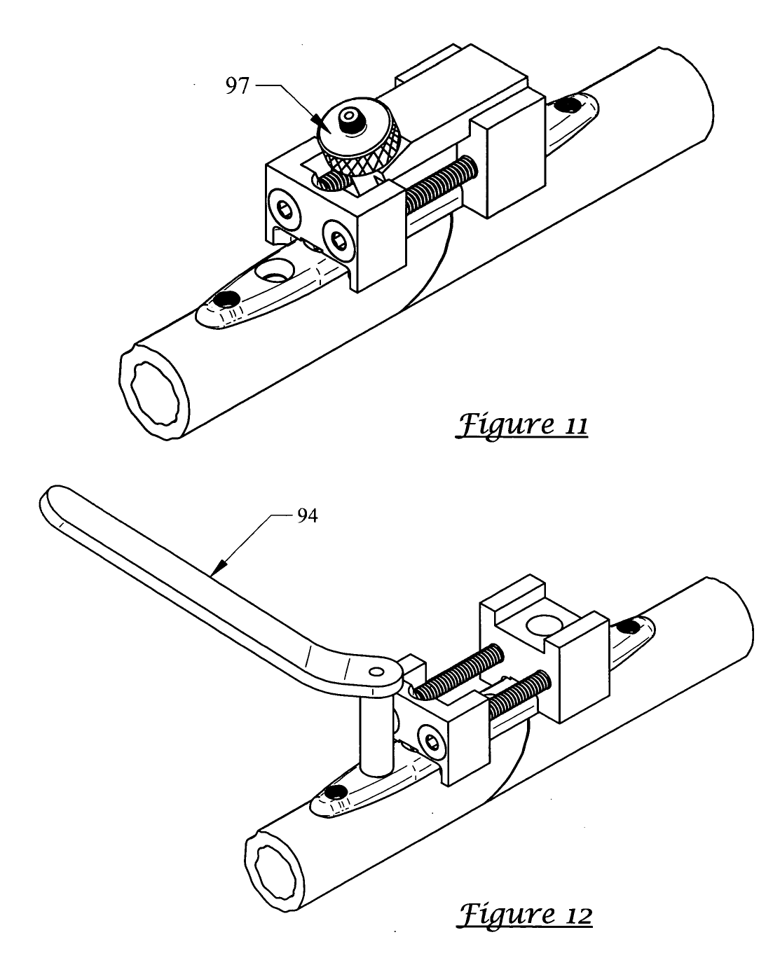

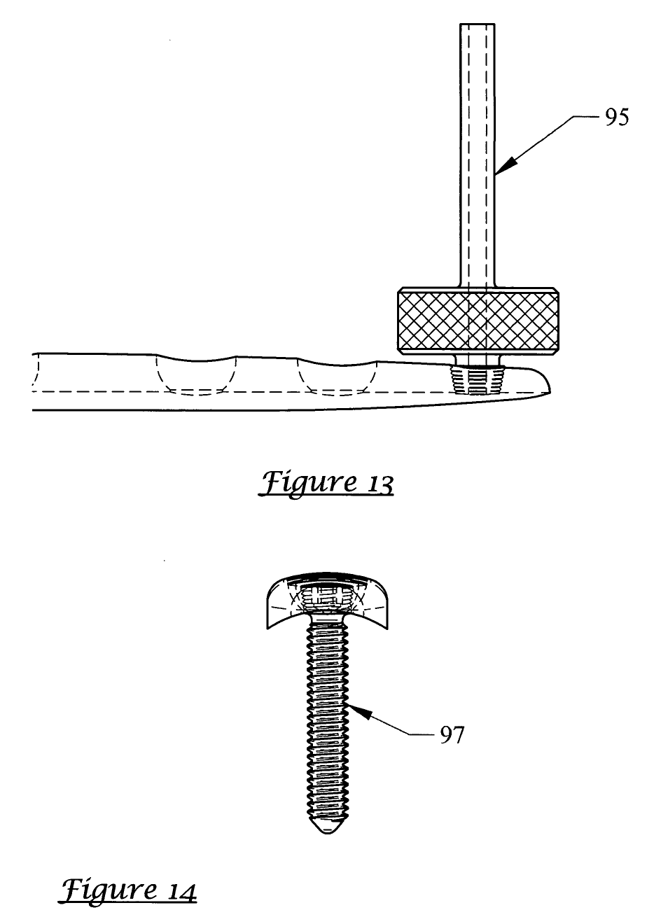

To prepare the bone plate for attachment, a pair of bone plate benders 72, illustrated in FIG. 8, can be used. In particular, the bone plate slides into bone plate openings 80 in each plate bender and then the surgeon carefully bends the bone plate into the appropriate shape for the ulna to be repaired. As shown in FIG. 9, the plate is secured to the ulna through opening 18 and a low profile compression device is applied through openings 22 and 26 with temporary screws that are 4 millimeters longer (not shown). Longitudinal compression screws 91 and 92 are tightened, bringing the bone ends together. As shown in FIG. 10, a low profile angle drill guide 93 is applied and a 22.5 degree hole is made through bit hole 96 with a 2.7 mm drill bit. As illustrated in FIG. 11, a combination drill bushing 95 is used to drill the far cortex with a 2.0 mm drill bit. The cortex is then tapped and a 2.7 mm cortical interfragmentary lag screw is inserted. As illustrated in FIG. 12, opening 30 is drilled with a hand held drill guide 94 and a 3.5 mm cortical is screwed into the opening. As seen in FIG. 13, the combination drill guide bushing 95 is threaded into hole 14 and the 2.0 mm drill hole is made. As seen in FIGS. 2 and 14, the 2.7 to 3.0 mm threaded locking screw 97 is inserted with a 2.0 mm hex screw driver (not shown). This process is repeated for hole 16.

It is therefore intended that the foregoing detailed description be regarded as illustrative rather than limiting, and that it be understood that it is the following claims, including all equivalents, that are intended to define the spirit and scope of this invention.

What is claimed is:

1. An improved slotted plate for use in an osteotomy procedure, the improved plate comprising:
a tapered proximal end portion, a tapered distal end portion, and a longitudinal portion extending therebetween, the longitudinal portion having a top surface and a bottom surface, the bottom surface having a female contour adapted to mate with the male contour of a bone surface, wherein the top and bottom surfaces are spaced apart a maximum distance between 0.119 in. and 0.113 in.;
a slot extending from the top surface to the bottom surface;
and a plurality of openings extending from the top surface to the bottom surface, wherein at least a first opening forms an inner periphery, the inner periphery having a female thread adapted to receive a machine-threaded locking screw.

2. The improved slotted plate of claim 1, wherein a second opening forms an inner periphery, the inner periphery have a female thread adapted to receive a machine-threaded locking screw.

3. The improved slotted plate of claim 2, wherein the first opening is disposed within the proximal end portion and the second opening is disposed within the distal end portion.

4. The improved slotted plate of claim 3, further comprising a plurality of openings having a smooth inner periphery, wherein each opening is adapted to receive a conventional cortical bone screw.

5. The improved slotted plate of claim 4, further comprising a ramped surface extending between the proximal end portion and the longitudinal portion, and a second ramped surface extending between the distal end portion and the longitudinal portion, wherein the longitudinal portion is gently curved.

6. The improved slotted plate of claim 5, wherein the improved plate is machined from titanium.

7. The improved slotted plate of claim 5, wherein the improved plate is machined from stainless steel.

8. The improved slotted plate of claim 5, wherein the improved plate is formed from high density plastic.

9. The improved slotted plate of claim 5, wherein the improved plate is formed from a biodegradable material.

10. The improved slotted plate of claim 9, wherein the improved biodegradable material is a copolymer.

11. The improved slotted plate of claim 1, wherein the plurality of openings comprises five openings.

12. The improved slotted plate of claim 1, wherein the plurality of openings consists of six openings.

13. The improved slotted plate of claim 1, wherein each of the plurality of openings forms an inner periphery having a female thread adapted to receive a machine-threaded locking screw.

14. The improved slotted plate of claim 13, further comprising a second plurality of openings extending from the top surface to the bottom surface, each of the second plurality of openings having a chamfered inner periphery.

15. The improved slotted plate of claim 14, wherein the slot comprises a reinforced portion.

16. The improved slotted plate of claim 15, further comprising a ramped portion disposed at the distal end portion and a second ramped portion disposed at the proximal end portion.

17. An improved slotted plate for use in an osteotomy procedure, the improved plate comprising:
a tapered proximal end portion, a tapered distal end portion, and a longitudinal portion extending therebetween, the end portions and the longitudinal portion having a top surface and a bottom surface, wherein the maximum distance between the top surface and the bottom surface is between 0.119 in. and 0.113 in.;
a first opening extending from the top surface to the bottom surface of the tapered proximal end portion, the first opening having a female thread adapted to receive a machine threaded locking screw;
a second opening extending from the top surface to the bottom surface of the tapered proximal end portion, the second opening being adapted to receive a cortical screw;
a third opening extending from the top surface to the bottom surface of the tapered proximal end portion, the third opening being adapted to receive a cortical screw;
a fourth opening extending from the top surface to the bottom surface of the tapered distal end portion, the fourth opening being adapted to receive a cortical screw;
a fifth opening extending from the top surface to the bottom surface of the tapered distal end portion, the fifth opening having a female thread adapted to receive a machine threaded locking screw, wherein the second, third, and fourth openings have a larger diameter than the first and fifth openings; and
a reinforced elongate opening extending from the top surface to the bottom surface of the longitudinal portion.

18. A surgical kit for performing an oblique ulnar osteotomy, comprising:
an improved slotted plate, comprising:
a tapered proximal end portion, a tapered distal end portion, and a longitudinal portion extending therebetween, the longitudinal portion having a top surface and a bottom surface, the bottom surface having a female contour adapted to mate with the male contour of a bone surface, wherein the top and bottom surfaces are spaced apart a maximum distance between 0.119 in. and 0.113 in.;
a slot extending from the top surface to the bottom surface; and
a plurality of openings extending from the top surface to the bottom surface, wherein at least a first opening forms an inner periphery, the inner periphery having a female thread adapted to receive a machine-threaded locking screw;
a saw blade configured for use with a saw selected from the group of a pneumatic saw, an electric saw, and an electric/pneumatic saw;
a low profile saw guide configured to be secured to an ulnar surface, the saw guide having a plurality of slots, the slots being adapted to receive the saw blade;
a straight drill guide having drill guide openings corresponding with at least two of the openings of the saw guide;
a low-profile compression device; and
an equipment tray adapted to accommodate the slotted plate, the saw guide, the straight drill guide, and the compression device.

19. The surgical kit of claim 18, further comprising a plurality of locking screws for securing the improved slotted plate to a long bone, the locking screws being configured to mate with a screw driver.

20. The surgical kit of claim 19 further comprising a screw driver configured to mate with the locking screws.

21. The surgical kit of claim 20, further comprising an angled drill guide configured for use with the low profile compression device.

22. The surgical kit of claim 21, wherein the angled drill guide includes an interior angle of 22.5 degrees.

23. The surgical kit of claim 22, further comprising a combination drill guide having a drill bushing and a smooth end configured for use with the angled drill guide.

24. The surgical kit of claim 23, further comprising a pair of plate benders, each plate bender comprising a handle portion; a first opening having a first interior dimension and a second portion having a second interior dimension, the opening being adapted to receive the improved slotted plate.

25. The surgical kit of claim 24, further comprising a combination drill guide having a drill bushing and a threaded end configured to mate with the female thread of the improved slotted plate.

26. The surgical kit of claim 25 further comprising a hand held drill guide configured to guide a drill through the improved bone plate and a bone.
